(19) United States
(12) Patent Application Publication
(10) Pub. No.: US 2024/0245304 A1
(43) Pub. Date: Jul. 25, 2024

(54) SYSTEM AND METHOD FOR LEARNING RELATIONSHIPS BETWEEN PHYSIOLOGICAL SIGNALS AND PSYCHOLOGICAL STATE TO PREDICT INDIVIDUAL BEHAVIOR

(71) Applicant: TOYOTA RESEARCH INSTITUTE, INC., Los Altos, CA (US)

(72) Inventors: Shabnam HAKIMI, San Francisco, CA (US); Charlene WU, San Francisco, CA (US); Yanxia ZHANG, Foster City, CA (US)

(73) Assignees: TOYOTA RESEARCH INSTITUTE, INC., Los Altos, CA (US); TOYOTA JIDOSHA KABUSHIKI KAISHA, Aichi-Ken (JP)

(21) Appl. No.: 18/099,187

(22) Filed: Jan. 19, 2023

(51) Int. Cl. A61B 5/0205; A61B 5/00

(52) U.S. Cl. CPC A61B 5/0205; A61B 5/7275

(57) ABSTRACT

A method for learned behavior prediction is described. The method includes receiving physiological data from a plurality of different modalities. The method also includes grouping the received physiological data by corresponding, similar behaviors. The method further includes learning, by a transfer function learner, a shared latent space, in which embeddings from the different modalities are grouped according to the similar behaviors. The method also includes utilizing, by a behavior prediction engine, a trained, transfer function learner to predict an individual's future behavior based on an input of physiological data and a user-specified prediction model.

### BACKGROUND

### Field

Certain aspects of the present disclosure generally relate to machine learning and, more particularly, to a system and method for learning relationships of physiological signals as proxies for a psychological state to predict individual behavior.

### Background

Predicting human behavior is important for many domains, including election forecasting, market analytics, public policy support, insurance markets, and medical choices. Physiological signals can provide insight into what is happening in an unobservable mind (a psychological state), which may be used to predict human behavior. Research efforts, however, are hampered by a limited understanding of the relationships between different physiological signals as well as a limited utility of theoretical models for effecting and predicting individual human behavior.

Individuals make choices for various reasons. These choices may be significantly influenced by a psychological state of the individual. Unfortunately, effectively determining the psychological state of the individual is not an exact science. A behavior prediction system for 1) learning the relationships between physiological signals as proxies for a psychological state, and 2) using the learned representation to predict individual behavior (e.g., economic decisions), is desired.

### SUMMARY

A method for learned behavior prediction is described. The method includes receiving physiological data from a plurality of different modalities. The method also includes grouping the received physiological data by corresponding, similar behaviors. The method further includes learning, by a transfer function learner, a shared latent space, in which embeddings from the different modalities are grouped according to the similar behaviors. The method also includes utilizing, by a behavior prediction engine, a trained, transfer function learner to predict an individual's future behavior based on an input of physiological data and a user-specified prediction model.

A non-transitory computer-readable medium having program code recorded thereon for learned behavior prediction is described. The program code is executed by a processor. The non-transitory computer-readable medium includes program code to receive a physiological data from a plurality of different modalities. The non-transitory computer-readable medium also includes program code to group the physiological data by corresponding, similar behaviors. The non-transitory computer-readable medium further includes program code to learn a shared latent space, in which embeddings from the different modalities are grouped according to the similar behaviors using a transfer function learner. The non-transitory computer-readable medium also includes program code to utilize a trained, transfer function learner to predict an individual's future behavior based on an input of physiological data and a user-specified prediction model using a behavior prediction engine.

A system for learned behavior prediction is described. The system includes a physiological data collection module to receive a physiological data from a plurality of different modalities. The system also includes a physiological behavior grouping module to group the physiological data by corresponding, similar behaviors. The system further includes a transfer function learner to learn a shared latent space, in which embeddings from the different modalities are grouped according to the similar behaviors. The system also includes a behavior prediction engine to utilize a trained, transfer function learner to predict an individual's future behavior based on an input of physiological data and a user-specified prediction model.

This has outlined, rather broadly, the features and technical advantages of the present disclosure in order that the detailed description that follows may be better understood. Additional features and advantages of the present disclosure will be described below. It should be appreciated by those skilled in the art that this present disclosure may be readily utilized as a basis for modifying or designing other structures for carrying out the same purposes of the present disclosure. It should also be realized by those skilled in the art that such equivalent constructions do not depart from the teachings of the present disclosure as set forth in the appended claims. The novel features, which are believed to be characteristic of the present disclosure, both as to its organization and method of operation, together with further objects and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

The features, nature, and advantages of the present disclosure will become more apparent from the detailed description set forth below when taken in conjunction with the drawings in which like reference characters identify correspondingly throughout.

FIG. 1 illustrates an example implementation of designing a neural network using a system-on-a-chip (SOC) for a learned behavior prediction system, in accordance with aspects of the present disclosure.

FIG. 2 is a block diagram illustrating an exemplary software architecture that may modularize artificial intelligence (AI) functions for a learned behavior prediction system, according to aspects of the present disclosure.

FIG. 3 is a diagram illustrating a hardware implementation for a learned behavior prediction system, according to aspects of the present disclosure.

FIG. 4 is a block diagram illustrating a learned behavior prediction system, in accordance with aspects of the present disclosure.

FIG. 5 is a flowchart illustrating a method for learned behavior prediction, according to aspects of the present disclosure.

### DETAILED DESCRIPTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. It will be apparent to those skilled in the art, however, that these concepts may be practiced without these specific details. In some instances, well-known structures and components are shown in block diagram form in order to avoid obscuring such concepts.

Based on the teachings, one skilled in the art should appreciate that the scope of the present disclosure is intended to cover any aspect of the present disclosure, whether implemented independently of or combined with any other aspect of the present disclosure. For example, an apparatus may be implemented or a method may be practiced using any number of the aspects set forth. In addition, the scope of the present disclosure is intended to cover such an apparatus or method practiced using other structure, functionality, or structure and functionality in addition to, or other than the various aspects of the present disclosure set forth. It should be understood that any aspect of the present disclosure disclosed may be embodied by one or more elements of a claim.

Although particular aspects are described herein, many variations and permutations of these aspects fall within the scope of the present disclosure. Although some benefits and advantages of the preferred aspects are mentioned, the scope of the present disclosure is not intended to be limited to particular benefits, uses, or objectives. Rather, aspects of the present disclosure are intended to be broadly applicable to different technologies, system configurations, networks, and protocols, some of which are illustrated by way of example in the figures and in the following description of the preferred aspects. The detailed description and drawings are merely illustrative of the present disclosure, rather than limiting the scope of the present disclosure being defined by the appended claims and equivalents thereof.

Researchers in multiple disciplines (e.g., psychology, neuroscience, marketing) rely on physiological signals to build models of psychological processes that should, theoretically, be related to behavior. For example, these psychological processes may refer to a series of steps that occur in a regular way to attain a change in behavior, emotion, or thought. These psychological processes may also involve sensation, perception, attention, learning, memory, language, and motivation. Often, researchers have limited data from limited sensors. For example, a researcher may have heart rate data but no brain data. Without a way of estimating what the brain response might be in this case, researchers cannot make use of the insights that the brain may offer into the underlying psychological process. The same is true for any other physiological sensors that may not be available to the researcher.

In behavioral science, physiological signals are routinely used to infer an individual's psychological state. A variety of techniques are used to measure physiological signals, including brain response, eye movements, and heart rate. These physiological signals can provide insight into what is happening in the unobservable mind. Research efforts, however, are hampered by a limited understanding of the relationships between these different physiological signals as well as a limited utility of theoretical models. In particular, these research efforts do not allow for multimodal learning of shared representations between psychological processes and different physiological signals for predicting related behavior.

Predicting human behavior is important for many domains, including election forecasting, market analytics, public policy support, insurance markets, and medical choices. Physiological signals can provide insight into what is happening in an unobservable mind (a psychological state), which may be used to predict human behavior. Research efforts, however, are hampered by a limited understanding of the relationships between different physiological signals as well as a limited utility of theoretical models for effecting and predicting individual human behavior. Yet, individuals make choices for various reasons. These choices may be significantly influenced by a psychological state of the individual. Unfortunately, effectively determining the psychological state of the individual is not an exact science, which prohibits effective prediction of individual choices.

Some aspects of the present disclosure are directed to a learned behavior prediction system for 1) learning the relationships between the physiological signals as proxies for a psychological state and 2) using the learned representation to predict individual behavior (e.g., economic decisions). These aspects of the present disclosure extend an approach to multi-sensor physiological data by establishing a method that is applied to enhance inference on psychological processes for significantly improving behavior prediction. In some aspects of the present disclosure, a behavior prediction engine receives the following inputs: (1) a user-specified prediction model (e.g., logistic regression) and (2) any available physiological data associated with the behavior of interest. In this example, the behavior prediction engine uses these data to predict future user behavior.

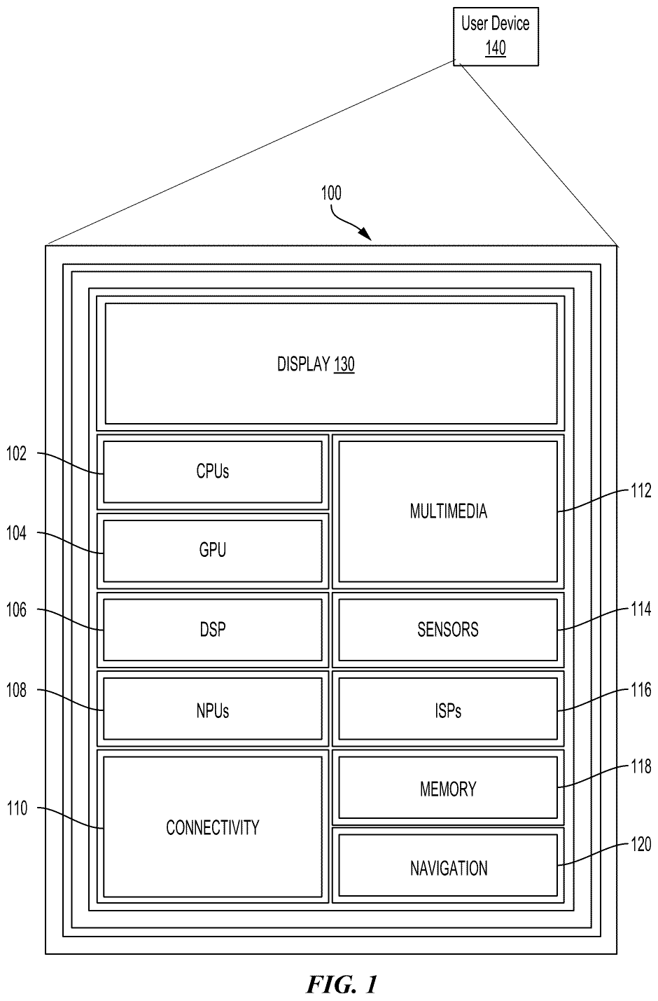

FIG. 1 illustrates an example implementation of the aforementioned system and method for a learned behavior prediction system using a system-on-a-chip (SOC) 100, according to aspects of the present disclosure. The SOC 100 may include a single processor or multi-core processors (e.g., a central processing unit (CPU) 102), in accordance with certain aspects of the present disclosure. Variables (e.g., neural signals and synaptic weights), system parameters associated with a computational device (e.g., neural network with weights), delays, frequency bin information, and task information may be stored in a memory block. The memory block may be associated with a neural processing unit (NPU) 108, a CPU 102, a graphics processing unit (GPU) 104, a digital signal processor (DSP) 106, a dedicated memory block 118, or may be distributed across multiple blocks. Instructions executed at a processor (e.g., CPU 102) may be loaded from a program memory associated with the CPU 102 or may be loaded from the dedicated memory block 118.

The SOC 100 may also include additional processing blocks configured to perform specific functions, such as the GPU 104, the DSP 106, and a connectivity block 110, which may include fourth generation long term evolution (4G LTE) connectivity, unlicensed Wi-Fi connectivity, USB connectivity, Bluetooth® connectivity, and the like. In addition, a multimedia processor 112 in combination with a display 130 may, for example, select a control action, according to the display 130 illustrating a view of a user device.

In some aspects, the NPU 108 may be implemented in the CPU 102, DSP 106, and/or GPU 104. The SOC 100 may further include sensors 114, image signal processors (ISPs) 116, and/or navigation 120, which may, for instance, include a global positioning system. The SOC 100 may be based on an Advanced Risk Machine (ARM) instruction set or the like. In another aspect of the present disclosure, the SOC 100 may be a server computer in communication with a user device 140. In this arrangement, the user device 140 may include a processor and other features of the SOC 100.

In this aspect of the present disclosure, instructions loaded into a processor (e.g., CPU 102) or the NPU 108 of the user device 140 may include code to learn relationships of physiological signals as proxies for psychological states to predict individual behavior. The instructions loaded into a processor (e.g., NPU 108) may also include code to receive physiological data from a plurality of different modalities. The instructions loaded into a processor (e.g., NPU 108) may also include code to group similar behaviors from the received physiological data. The instructions loaded into a processor (e.g., NPU 108) may also include code to learn, by a transfer function learner, a shared latent space, in which embeddings from the different modalities are grouped according to the grouping of the similar behaviors. The instructions loaded into a processor (e.g., NPU 108) may also include code to utilize, by a behavior prediction engine, a trained, transfer function learner to predict an individual's future behavior based on an input of physiological data and a user-specified prediction model.

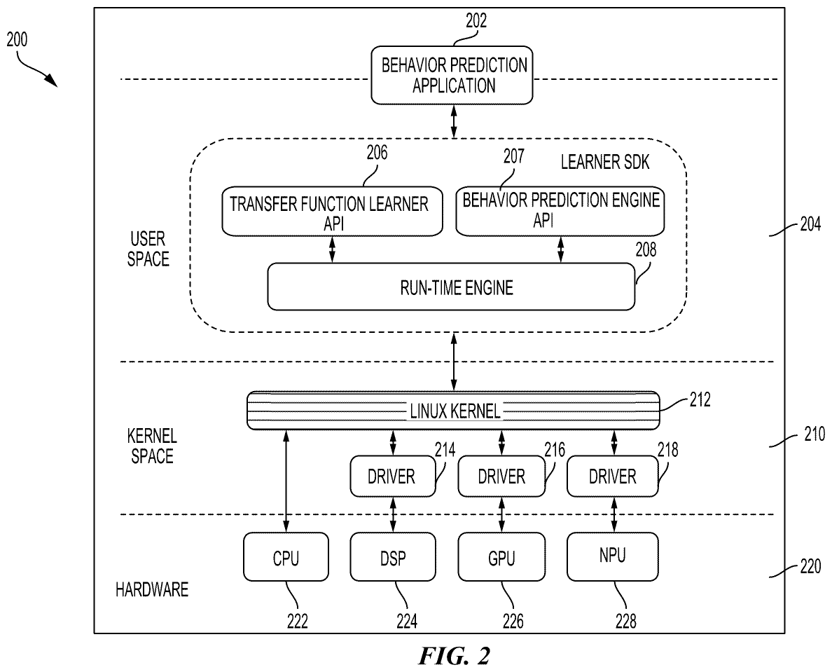

FIG. 2 is a block diagram illustrating a software architecture 200 that may modularize artificial intelligence (AI) functions for learning relationships of physiological signals as proxies for psychological states to predict an individual's future behavior, according to aspects of the present disclosure. Using the architecture, a behavior prediction application 202 may be designed such that it may cause various processing blocks of an SOC 220 (for example a CPU 222, a DSP 224, a GPU 226, and/or an NPU 228) to perform supporting computations during run-time operation of the behavior prediction application 202. Although FIG. 2 describes the software architecture 200 for a learned behavior prediction system, it should be recognized that the learned behavior prediction system is applicable to any type of decision or individual activity.

The behavior prediction application 202 may be configured to call functions defined in a user space 204 that may, for example, provide for learned behavior prediction services. The behavior prediction application 202 may make a request for compiled program code associated with a library defined in a transfer function learner application programming interface (API) 206. The transfer function learner API 206 is configured to learn a shared latent space, in which embeddings from the different modalities are grouped according to a grouping of the similar behaviors. In some aspects of the present disclosure, given multiple input physiological data streams (e.g., brain responses, eye gaze, heart rate), the transfer function learner API 206 learns a shared latent space where the embeddings from different modalities are grouped by similar behaviors. This learning process may be performed using an embedding learning step, followed by an association and transfer step, as described in further detail below.

The behavior prediction application 202 may further make a request for compiled program code associated with a library defined in a behavior prediction engine API 207. The behavior prediction engine API 207 is configured to utilize a trained, transfer function learner API 206 to predict an individual's future behavior based on an input of physiological data and a user-specified prediction model. In some aspects of the present disclosure, the behavior prediction engine API 207 leverages a pre-trained, transfer function learner API 206 to predict an individual's future behavior even when provided a limited number of inputs. The behavior prediction engine API 207 may receive the following inputs: (1) a user-specified prediction model (e.g., logistic regression) and (2) any available physiological data associated with the behavior of interest. In this example, the behavior prediction engine API 207 uses these data to predict behavior in unseen data (e.g., test set), as described in further detail below:

A run-time engine 208, which may be compiled code of a run-time framework, may be further accessible to the behavior prediction application 202. The behavior prediction application 202 may cause the run-time engine 208 to send a signal to an operating system 210, such as a Linux Kernel 212, running on the SOC 220. FIG. 2 illustrates the Linux Kernel 212 as software architecture for learned behavior prediction system. It should be recognized, however, that aspects of the present disclosure are not limited to this exemplary software architecture. For example, other kernels may provide the software architecture to support the learned behavior prediction functionality.

The operating system 210, in turn, may cause a computation to be performed on the CPU 222, the DSP 224, the GPU 226, the NPU 228, or some combination thereof. The CPU 222 may be accessed directly by the operating system 210, and other processing blocks may be accessed through a driver, such as drivers 214-218 for the DSP 224, for the GPU 226, or for the NPU 228. In the illustrated example, the deep neural network may be configured to run on a combination of processing blocks, such as the CPU 222 and the GPU 226, or may be run on the NPU 228, if present.

Predicting human behavior is important for many domains, including election forecasting, market analytics, public policy support, insurance markets, and medical choices. Physiological signals can provide insight into what is happening in an unobservable mind (a psychological state), which may be used to predict human behavior. Research efforts, however, are hampered by a limited understanding of the relationships between different physiological signals as well as a limited utility of theoretical models for effecting and predicting individual human behavior. Yet, individuals make choices for various reasons. These choices may be significantly influenced by a psychological state of the individual. Unfortunately, effectively determining the psychological state of the individual is not an exact science, which prohibits effective prediction of individual choices.

Some aspects of the present disclosure are directed to a learned behavior prediction system for 1) learning the relationships between the physiological signals as proxies for a psychological state, and 2) using the learned representation to predict individual behavior (e.g., economic decisions). These aspects of the present disclosure extend an approach to multi-sensor physiological data by establishing a method that is applied to enhance inference on psychological processes for significantly improving behavior prediction. In some aspects of the present disclosure, a behavior prediction engine receives the following inputs: (1) a user-specified prediction model (e.g., logistic regression) and (2) any available physiological data associated with the behavior of interest. In this example, the behavior prediction engine uses these data to predict future user behavior.

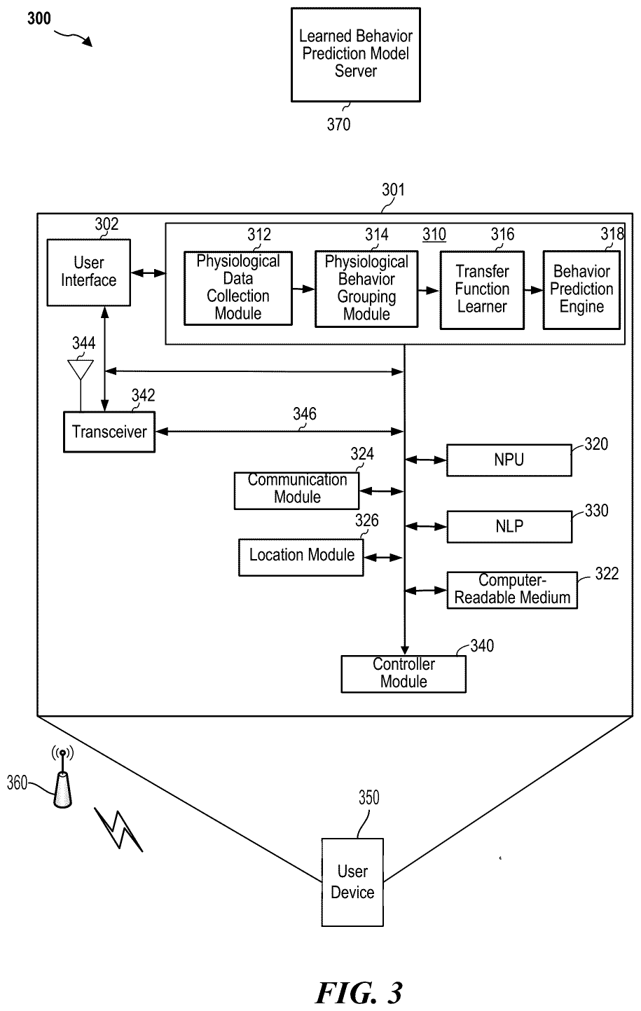

FIG. 3 is a diagram illustrating a hardware implementation for a learned behavior prediction system 300, according to aspects of the present disclosure. The learned behavior prediction system 300 may be configured to identify an individual's future behavior that a user desires to predict and relevant physiological and psychological signals influencing the individual's future behavior. In some aspects of the present disclosure, given multiple input physiological data streams (e.g., brain responses, eye gaze, heart rate), the learned behavior prediction system 300 learns a shared latent space where the embeddings from different modalities are grouped by similar behaviors. This learning process may be performed using an embedding learning step, followed by an association and transfer step, as described in further detail below:

In some aspects of the present disclosure, the learned behavior prediction system 300 leverages a pre-trained, transfer function learner to predict an individual's future behavior even when provided a limited number of inputs by relying on inferred physiological data. The learned behavior prediction system 300 may receive the following inputs: (1) a user-specified prediction model (e.g., logistic regression) and (2) any available physiological data associated with the behavior of interest. In this example, the learned behavior prediction system 300 uses these data to predict behavior in unseen data (e.g., test set), as described in further detail below.

The learned behavior prediction system 300 includes a transfer function and behavior prediction system 301 and a learned behavior prediction model server 370 in this aspect of the present disclosure. The transfer function and behavior prediction system 301 may be a component of a user device 350. The user device 350 may be a cellular phone (e.g., a smart phone), a personal digital assistant (PDA), a wireless modem, a wireless communications device, a handheld device, a laptop computer, a cordless phone, a wireless local loop (WLL) station, a tablet, a camera, a gaming device, a netbook, a Smartbook, an Ultrabook, a medical device or equipment, biometric sensors/devices, wearable devices (smart watches, smart clothing, smart glasses, smart wrist bands, smart jewelry (e.g., smart ring, smart bracelet)), an entertainment device (e.g., a music or video device, or a satellite radio), a global positioning system device, or any other suitable device that is configured to communicate via a wireless or wired medium.

The learned behavior prediction model server 370 may connect to the user device 350 for providing future behavior predictions. For example, the learned behavior prediction server 370 may receive an individual's future behavior that a user desires to predict and relevant physiological and psychological signals influencing the individual's future behavior. Given multiple input physiological data streams (e.g., brain responses, eye gaze, heart rate), the learned behavior prediction model server 370 may provide a machine learning (ML)-based transfer function that learns a shared latent space where the embeddings from different modalities are grouped by similar behaviors. The learned behavior prediction model server 370 may also transmit a predicted future behavior estimated using a behavior prediction engine that is displayed by the user device 350.

The transfer function and behavior prediction system 301 may be implemented with an interconnected architecture, represented generally by an interconnect 346. The interconnect 346 may include any number of point-to-point interconnects, buses, and/or bridges depending on the specific application of the transfer function and behavior prediction system 301 and the overall design constraints. The interconnect 346 links together various circuits including one or more processors and/or hardware modules, represented by a user interface 302, a behavior prediction module 310, a neutral network processor (NPU) 320, a computer-readable medium 322, a communication module 324, a location module 326, a natural language processor (NLP) 330, and a controller module 340. The interconnect 346 may also link various other circuits such as timing sources, peripherals, voltage regulators, and power management circuits, which are well known in the art, and, therefore, will not be described any further.

The transfer function and behavior prediction system 301 includes a transceiver 342 coupled to the user interface 302, the behavior prediction module 310, the NPU 320, the computer-readable medium 322, the communication module 324, the location module 326, the NLP 330, and the controller module 340. The transceiver 342 is coupled to an antenna 344. The transceiver 342 communicates with various other devices over a transmission medium. For example, the transceiver 342 may receive commands via transmissions from another user or a connected device. In this example, the transceiver 342 may receive/transmit information for the behavior prediction module 310 to/from connected devices within the vicinity of the user device 350.

The transfer function and behavior prediction system 301 includes the NPU 320 coupled to the computer-readable medium 322. The NPU 320 performs processing, including the execution of software stored on the computer-readable medium 322 to provide a neural network model for transfer function learning and behavior prediction functionality according to the present disclosure. The software, when executed by the NPU 320, causes the transfer function and behavior prediction system 301 to perform the various functions described for learning relationships of physiological signals as proxies for psychological states to predict an individual's future behavior through the user device 350, or any of the modules (e.g., 310, 324, 326, and/or 340). The computer-readable medium 322 may also be used for storing data that is manipulated by the NLP 330 when executing the software to analyze user communications.

The location module 326 may determine a location of the user device 350. For example, the location module 326 may use a global positioning system (GPS) to determine the location of the user device 350. The location module 326 may implement a dedicated short-range communication (DSRC)-compliant GPS unit. A DSRC-compliant GPS unit includes hardware and software to make the autonomous vehicle 350 and/or the location module 326 compliant with the following DSRC standards, including any derivative or fork thereof: EN 12253:2004 Dedicated Short-Range Communication-Physical layer using microwave at 5.8 GHz (review); EN 12795:2002 Dedicated Short-Range Communication (DSRC)-DSRC Data link layer: Medium Access and Logical Link Control (review); EN 12834:2002 Dedicated Short-Range Communication-Application layer (review); EN 13372:2004 Dedicated Short-Range Communication (DSRC)-DSRC profiles for RTTT applications (review); and EN ISO 14906:2004 Electronic Fee Collection-Application interface.

The communication module 324 may facilitate communications via the transceiver 342. For example, the communication module 324 may be configured to provide communication capabilities via different wireless protocols, such as 6G, 5G new radio (NR), Wi-Fi, long term evolution (LTE), 4G, 3G, etc. The communication module 324 may also communicate with other components of the user device 350 that are not modules of the transfer function and behavior prediction system 301. The transceiver 342 may be a communications channel through a network access point 360. The communications channel may include DSRC, LTE, LTE-D2D, mmWave, Wi-Fi (infrastructure mode), Wi-Fi (ad-hoc mode), visible light communication, TV white space communication, satellite communication, full-duplex wireless communications, or any other wireless communications protocol such as those mentioned herein.

The transfer function and behavior prediction system 301 also includes the NLP 330 to receive and analyze language from a data log of choice communications to transfer functions for predicting an individual's future behavior. In some aspects of the present disclosure, natural language processing of the NLP 330 is applied to a data log for extracting terms from an individual's choices, regarding, for example, the effects of social distance and personal distance on the individual's choices. In aspects of the present disclosure, the NLP 330 is used if the communications are conducted in plain text.

The behavior prediction module 310 may be in communication with the user interface 302, the NPU 320, the computer-readable medium 322, the communication module 324, the location module 326, the NLP 330, the controller module 340, and the transceiver 342. In one configuration, the behavior prediction module 310 monitors physiological signals received from the user device 350 through the user interface 302. The user interface 302 allows the user to input available data, compute the learned representation using the transfer function learner, and use the outputs to (1) explore relationships between signals and (2) use available and inferred signals to make predictions about subsequent behavior. In some aspects of the present disclosure, the user interface 302 also includes an option to link to scientific research databases (e.g., PubMed, Google Scholar) that can provide the user with further learning opportunities.

As shown in FIG. 3, the behavior prediction module 310 includes a physiological data collection module 312, a physiological behavior grouping module 314, a transfer function learner 316, and a behavior prediction engine 318. The transfer function learner 316 and the behavior prediction engine 318 may be components of a same or different artificial neural network, such as a deep convolutional neural network (CNN). The transfer function learner 316 and the behavior prediction engine 318 are not limited to a CNN. The behavior prediction module 310 is configured to learn relationships of physiological signals as proxies for psychological states to predict an individual's future behavior and personal distance, according to aspects of the present disclosure.

This configuration of the behavior prediction module 310 includes the physiological data collection module 312 for receiving physiological data from different modalities (e.g., brain responses, eye gaze, heart rate). The behavior prediction module 310 also includes the physiological behavior grouping module 314 for grouping the received physiological data by corresponding, similar behaviors. The behavior prediction module 310 also includes the transfer function learner 316 to provide a machine learning (ML)-based transfer function that learns a shared latent space where embeddings from different modalities are grouped by similar behaviors. The behavior prediction module 310 further includes the behavior prediction engine 318 to utilize a trained, transfer function learner 316 to predict an individual's future behavior based on an input of physiological data and a user-specified prediction model.

As noted above, predicting human behavior is important for many domains, including election forecasting, market analytics, public policy support, insurance markets, and medical choices. Physiological signals can provide insight into what is happening in an unobservable mind (a psychological state), which may be used to predict human behavior. Research efforts, however, are hampered by a limited understanding of the relationships between different physiological signals as well as a limited utility of theoretical models for effecting and predicting individual human behavior. Yet, individuals make choices for various reasons. These choices may be significantly influenced by a psychological state of the individual. Unfortunately, effectively determining the psychological state of the individual is not an exact science, which prohibits effective prediction of individual choices.

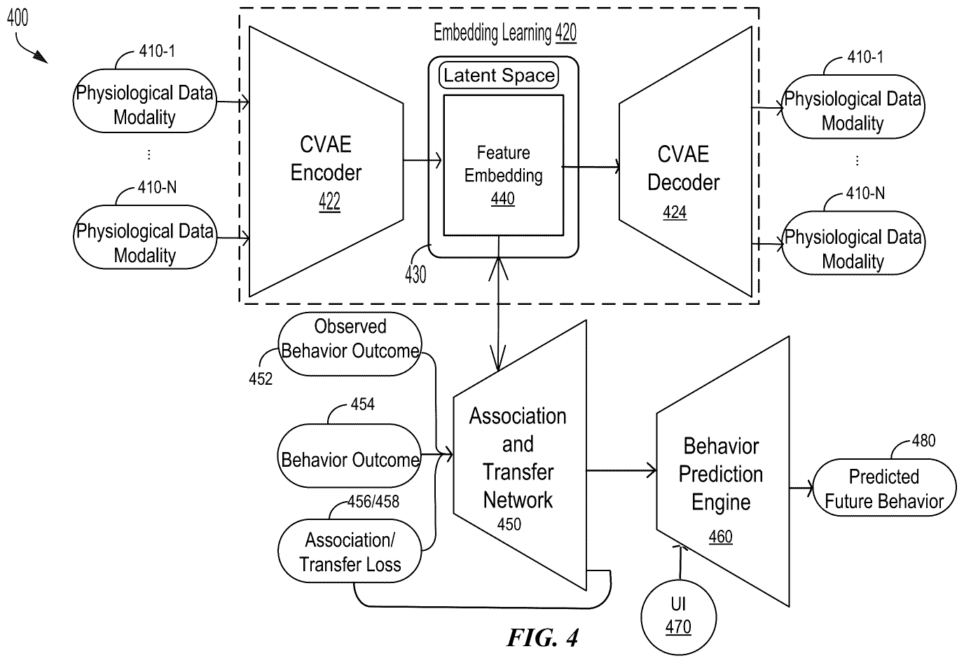

In some aspects of the present disclosure, given multiple input physiological data streams (e.g., brain responses, eye gaze, heart rate), the transfer function learner 316 learns a shared latent space where the embeddings from different modalities are grouped by similar behaviors. This learning process may be performed using an embedding learning step, followed by an association and transfer step. In some aspects of the present disclosure, the behavior prediction engine 318 leverages a pre-trained, transfer function learner 316 to predict an individual's future behavior even when provided a limited number of inputs. The behavior prediction engine 318 may receive the following inputs: (1) a user-specified prediction model (e.g., logistic regression) and (2) any available physiological data associated with the behavior of interest. In this example, the behavior prediction engine 318 uses these data to predict behavior in unseen data (e.g., test set), as shown in FIG. 4.

FIG. 4 is a block diagram illustrating a learned behavior prediction system 400, in accordance with aspects of the present disclosure. In this example, the learned behavior prediction system 400 assumes no strong priors about a psychological state, which allows for the possibility of learning relationships between physiology, psychology, and behavior that have not yet been well characterized in basic science. This approach differentiates aspects of the present disclosure from existing approaches for detecting user state. In particular, conventional emotion recognition models commonly include robust priors about signals associated with affective states (e.g., facial muscle activation associated with a discrete emotional state like happiness). Additionally, aspects of the present disclosure include a framework for using such diverse, multimodal signals to make predictions about a subsequent behavior. Such predictions can be further integrated with forecasting models.

As shown in FIG. 4, the learned behavior prediction system 400 includes a transfer function learner (e.g., embedding learning stage 420 and association and transfer network 450), a behavior prediction engine 460, and a user interface 470, in aspects of the present disclosure. In this example, in multiple input physiological data streams (e.g., brain responses, eye gaze, heart rate), the transfer function learner learns a shared latent space 430 where the embeddings from different modalities are grouped by similar behaviors.

In some aspects of the present disclosure, the embedding learning stage 420 provides an autoencoder network for receiving physiological data modalities (e.g., input data channels 410 (410-1, . . . , 410-N)) at an autoencoder (e.g., a conditional variational autoencoder (CVAE) encoder 422). For each modality (e.g., the input data channels 410), the CVAE encoder 422 is trained to convert the input data channels 410 into a feature embedding 440 (e.g., converted physiological data) within a shared latent space 430. In addition, a CVAE decoder 424 is trained to convert the feature embedding 440 back to the original modality (e.g., input data channels 410 (410-1, . . . , 410-N) of physiological data), which is referred to as a corresponding modality.

The transfer function learner further includes an association and transfer network 450, according to aspects of the present disclosure. As further illustrated in FIG. 4, the association and transfer network 450 is operated by using a joint association and transfer network that converts the feature embedding 440 from one modality to another modality for a given behavior outcome 454 (e.g., choice over a set of products). Additionally, the association and transfer network 450 is optimized with a transfer loss 458 that uses explicit cues obtained in a controlled environment to classify whether signals are obtained from the same event (observed behavior outcome 452). An association loss 456 uses implicit cues, such as time to group multimodality signal streams, whether synchronous or asynchronous. In some aspects of the present disclosure, the association and transfer network 450 is optimized by a sum of the association loss 456 and the transfer loss 458.

As further illustrated in FIG. 4, the learned behavior prediction system 400 includes the behavior prediction engine 460, according to aspects of the present disclosure. In some aspects of the present disclosure, the behavior prediction engine 460 leverages a pre-trained transfer function learner (e.g., the embedding learning stage 420 and the association and transfer network 450) to predict an individual's future behavior (e.g., a predicted future behavior 480), even when provided a limited number of inputs. Because the shared latent space 430 across modalities is previously learned, any input physiological signal can be converted to another modality. As a result, a user can, therefore, infer the predictive contribution of a signal that is unavailable to model. In some cases, if any of the modality signals is missing, the association and transfer network 450 and the shared latent space 430 can leverage to generate feature embedding 440 from other modalities that are available without additional labels and signals.

In some aspects of the present disclosure, the behavior prediction engine 460 takes as input (1) a user-specified prediction model (e.g., logistic regression) and (2) any available physiological data associated with a behavior of interest. In response, the behavior prediction engine 460 uses these data to generate the predicted future behavior 480, for example, in unseen data (e.g., a test set).

In some aspects of the present disclosure, the user interface (UI) 470 allows a user to input available data, compute a learned representation using the transfer function learner (e.g., the embedding learning stage 420 and the association and transfer network 450), and use the outputs to (1) explore relationships between signals and (2) use available and inferred signals to make predictions about subsequent behavior. In this example, the UI 470 includes an option to link to scientific research databases (e.g., PubMed, Google Scholar) that provide the user with further learning opportunities. For relationship exploration (1), the user can explore the space of inferred signals based on what they input to the transfer function learner. The UI 470 visualizes the signals, allowing the user to move back and forth between modalities and gain intuition not only for the structure of the representation space, but also for the model's confidence in the learned representation. The typical end user of this function is a researcher interested in better understanding the spatiotemporal relationships between signals. For example, this functionality can be used by scientists to conduct research oriented to discovery (e.g., what is the likely relationship between heart rate and the signal in a particular brain region?).

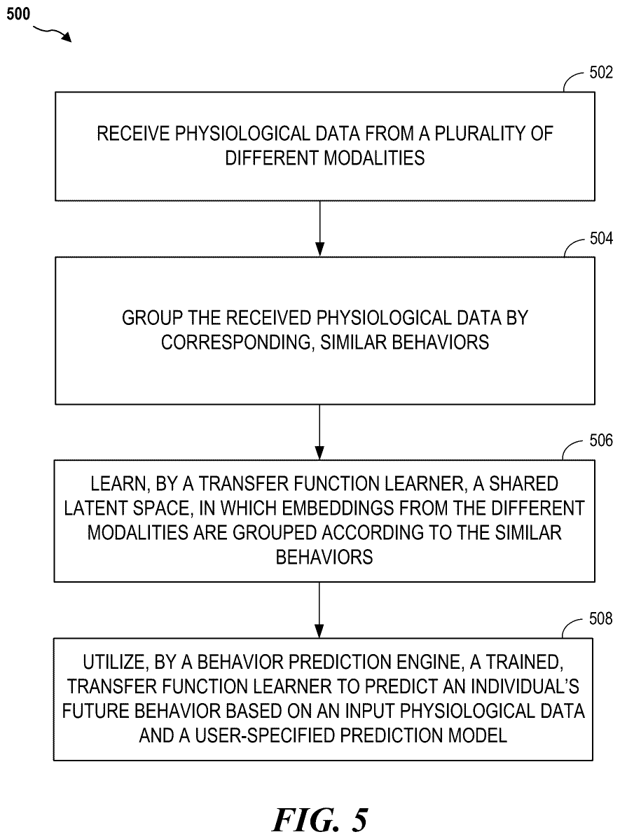

For predictions (2), the user may select from a library of models used to predict behaviors of interest (e.g., logistic regression to predict whether or not a person will purchase a product). The UI 470 provides a list of signals that can be inferred using the transfer function learner, given their inputs. Additionally, the user may select, based on their priors or the linked scientific articles, which inferred signals are likely useful for their question of interest. The UI 470 may provide the option to compute other permutations of model inputs (e.g., independent variables/predictors) that the user uses to compare to their chosen prediction model using model comparison metrics. For the dependent variable/outcome being predicted, the default is for the user to specify; however, the user may also choose from a library of outcomes of interest, for example, as further illustrated by the process of FIG. 5.

FIG. 5 is a flowchart illustrating a method for learned behavior prediction, according to aspects of the present disclosure. A method 500 of FIG. 5 begins at block 502, in which physiological data are received from a plurality of different modalities. For example, as described in FIG. 3, the behavior prediction module 310 includes the physiological data collection module 312 for receiving physiological data from different modalities (e.g., brain responses, eye gaze, heart rate). As shown in FIG. 4, the embedding learning stage 420 receives physiological data modalities from the input data channels 410 at the CVAE encoder 422.

Referring again to FIG. 5, at block 504, the received physiological data are grouped by corresponding, similar behaviors. For example, as shown in FIG. 3, the behavior prediction module 310 also includes the physiological behavior grouping module 314 for grouping the received physiological data by corresponding, similar behaviors. As shown in FIG. 4, the embedding learning stage 420 may group the physiological data received from the input data channels 410 by corresponding, similar behaviors.

At block 506, a transfer function learner learns a shared latent space, in which embeddings from the different modalities are grouped according to the similar behaviors. For example, as shown in FIG. 3, the behavior prediction module 310 also includes the transfer function learner 316 to provide a machine learning (ML)-based transfer function that learns a shared latent space where embeddings from different modalities are grouped by similar behaviors. As shown in FIG. 4, for each modality (e.g., the input data channels 410), the CVAE encoder 422 is trained to convert the input data channels 410 into a feature embedding 440 within a shared latent space 430. In addition, a CVAE decoder 424 is trained to convert the feature embedding 440 back to the original modality (e.g., the input data channels 410 of physiological data).

At block 508, a behavior prediction engine utilizes a trained, transfer function learner to predict an individual's future behavior based on an input physiological data and a user-specified prediction model. For example, as shown in FIG. 3, the behavior prediction module 310 further includes the behavior prediction engine 318 to utilize a trained, transfer function learner 316 to predict an individual's future behavior based on an input physiological data and a user-specified prediction model. The method 500 further includes exploring relationships between physiological data from the plurality of different modalities. The method 500 also includes predicting future behavior using an available physiological data and an inferred physiological data, for example, as shown in FIG. 4.

As shown in FIG. 4, the behavior prediction engine 460 leverages a pre-trained transfer function learner (e.g., the embedding learning stage 420 and the association and transfer network 450) to predict an individual's future behavior (the predicted future behavior 480, even when provided a limited number of inputs. For example, the behavior prediction engine 460 takes as input (1) a user-specified prediction model (e.g., logistic regression) and (2) any available physiological data associated with a behavior of interest. In response, the behavior prediction engine 460 uses these data to generate the predicted future behavior 480.

In the method 500, the relevant parameters may include an uncertainty parameter, a social distance parameter, and a personal distance parameter regarding the predicted choice. In the method 500, the user may include an educational researcher, and the choice comprises predicting a type of advice prospective students receive from their parents. The method 500 also includes suggesting generic function forms and common parameter values for the EGU model if the user does not have expert knowledge to make a selection. The method 500 also includes displaying the predicted choice by calculating, using the EGU model, a utility value as a function of an uncertainty parameter, a social distance parameter, and a personal distance parameter regarding the predicted choice. The method 500 also includes suggesting generic functions by providing a software default mode in which data values are selected from existing literature and/or from an internal database of previously run analyses, for example, as shown in FIGS. 3 and 4.

Some aspects of the present disclosure are directed to a learned behavior prediction system for 1) learning the relationships between the physiological proxies for psychological state and 2) using the learned representation to predict individual behavior (e.g., economic decisions). These aspects of the present disclosure extend an approach to multi-sensor physiological data by establishing a method that is applied to enhance inference on psychological processes for significantly improving behavior prediction. In some aspects of the present disclosure, a behavior prediction engine receives the following inputs: (1) a user-specified prediction model (e.g., logistic regression) and (2) any available physiological data associated with the behavior of interest. In this example, the behavior prediction engine uses these data to predict future user behavior.

The various operations of methods described above may be performed by any suitable means capable of performing the corresponding functions. The means may include various hardware and/or software component(s) and/or module(s), including, but not limited to, a circuit, an application-specific integrated circuit (ASIC), or processor. Generally, where there are operations illustrated in the figures, those operations may have corresponding counterpart means-plus-function components with similar numbering.

As used herein, the term “determining” encompasses a wide variety of actions. For example, “determining” may include calculating, computing, processing, deriving, investigating, looking up (e.g., looking up in a table, a database or another data structure), ascertaining, and the like. Additionally, “determining” may include receiving (e.g., receiving information), accessing (e.g., accessing data in a memory), and the like. Furthermore, “determining” may include resolving, selecting, choosing, establishing, and the like.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logical blocks, modules and circuits described in connection with the present disclosure may be implemented or performed with a processor configured according to the present disclosure, a digital signal processor (DSP), an ASIC, a field-programmable gate array signal (FPGA) or other programmable logic device (PLD), discrete gate or transistor logic, discrete hardware components, or any combination thereof designed to perform the functions described herein. The processor may be a microprocessor, but, in the alternative, the processor may be any commercially available processor, controller, microcontroller, or state machine specially configured as described herein. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the present disclosure may be embodied directly in hardware, in a software module executed by a processor, or in a combination of the two. A software module may reside in any form of storage medium that is known in the art. Some examples of storage media that may be used include random access memory (RAM), read-only memory (ROM), flash memory, erasable programmable read-only memory (EPROM), electrically erasable programmable read-only memory (EEPROM), registers, a hard disk, a removable disk, a CD-ROM, and so forth. A software module may comprise a single instruction, or many instructions, and may be distributed over several different code segments, among different programs, and across multiple storage media. A storage medium may be coupled to a processor such that the processor can read information from, and write information to, the storage medium. In the alternative, the storage medium may be integral to the processor.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

The functions described may be implemented in hardware, software, firmware, or any combination thereof. If implemented in hardware, an example hardware configuration may comprise a processing system in a device. The processing system may be implemented with a bus architecture. The bus may include any number of interconnecting buses and bridges depending on the specific application of the processing system and the overall design constraints. The bus may link together various circuits including a processor, machine-readable media, and a bus interface. The bus interface may connect a network adapter, among other things, to the processing system via the bus. The network adapter may implement signal processing functions. For certain aspects, a user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further.

The processor may be responsible for managing the bus and processing, including the execution of software stored on the machine-readable media. Examples of processors that may be specially configured according to the present disclosure include microprocessors, microcontrollers, DSP processors, and other circuitry that can execute software. Software shall be construed broadly to mean instructions, data, or any combination thereof, whether referred to as software, firmware, middleware, microcode, hardware description language, or otherwise. Machine-readable media may include, by way of example, RAM, flash memory, ROM, programmable read-only memory (PROM), EPROM, EEPROM, registers, magnetic disks, optical disks, hard drives, or any other suitable storage medium, or any combination thereof. The machine-readable media may be embodied in a computer-program product. The computer-program product may comprise packaging materials.

In a hardware implementation, the machine-readable media may be part of the processing system separate from the processor. However, as those skilled in the art will readily appreciate, the machine-readable media, or any portion thereof, may be external to the processing system. By way of example, the machine-readable media may include a transmission line, a carrier wave modulated by data, and/or a computer product separate from the device, all which may be accessed by the processor through the bus interface. Alternatively, or in addition, the machine-readable media, or any portion thereof, may be integrated into the processor, such as the case may be with cache and/or specialized register files. Although the various components discussed may be described as having a specific location, such as a local component, they may also be configured in various ways, such as certain components being configured as part of a distributed computing system.

The processing system may be configured with one or more microprocessors providing the processor functionality and external memory providing at least a portion of the machine-readable media, all linked together with other supporting circuitry through an external bus architecture. Alternatively, the processing system may comprise one or more neuromorphic processors for implementing the neuron models and models of neural systems described herein. As another alternative, the processing system may be implemented with an ASIC with the processor, the bus interface, the user interface, supporting circuitry, and at least a portion of the machine-readable media integrated into a single chip, or with one or more FPGAs, PLDs, controllers, state machines, gated logic, discrete hardware components, or any other suitable circuitry, or any combination of circuits that can perform the various functions described throughout this present disclosure. Those skilled in the art will recognize how best to implement the described functionality for the processing system depending on the particular application and the overall design constraints imposed on the overall system.

The machine-readable media may comprise a number of software modules. The software modules include instructions that, when executed by the processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a special purpose register file for execution by the processor. When referring to the functionality of a software module below, it will be understood that such functionality is implemented by the processor when executing instructions from that software module. Furthermore, it should be appreciated that aspects of the present disclosure result in improvements to the functioning of the processor, computer, machine, or other system implementing such aspects.

If implemented in software, the functions may be stored or transmitted over as one or more instructions or code on a non-transitory computer-readable medium. Computer-readable media include both computer storage media and communication media, including any medium that facilitates transfer of a computer program from one place to another. A storage medium may be any available medium that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Additionally, any connection is properly termed a computer-readable medium. For example, if the software is transmitted from a website, server, or other remote source using a coaxial cable, fiber optic cable, twisted pair, digital subscriber line (DSL), or wireless technologies such as infrared (IR), radio, and microwave, then the coaxial cable, fiber optic cable, twisted pair, DSL, or wireless technologies such as infrared, radio, and microwave are included in the definition of medium. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Thus, in some aspects computer-readable media may comprise non-transitory computer-readable media (e.g., tangible media). In addition, for other aspects, computer-readable media may comprise transitory computer-readable media (e.g., a signal). Combinations of the above should also be included within the scope of computer-readable media.

Thus, certain aspects may comprise a computer program product for performing the operations presented herein. For example, such a computer program product may comprise a computer-readable medium having instructions stored (and/or encoded) thereon, the instructions being executable by one or more processors to perform the operations described herein. For certain aspects, the computer program product may include packaging material.

Further, it should be appreciated that modules and/or other appropriate means for performing the methods and techniques described herein can be downloaded and/or otherwise obtained by a user terminal and/or base station as applicable. For example, such a device can be coupled to a server to facilitate the transfer of means for performing the methods described herein. Alternatively, various methods described herein can be provided via storage means (e.g., RAM, ROM, a physical storage medium such as a CD or floppy disk, etc.), such that a user terminal and/or base station can obtain the various methods upon coupling or providing the storage means to the device. Moreover, any other suitable technique for providing the methods and techniques described herein to a device can be utilized.

It is to be understood that the claims are not limited to the precise configuration and components illustrated above. Various modifications, changes, and variations may be made in the arrangement, operation, and details of the methods and apparatus described above without departing from the scope of the claims.

What is claimed is:

1. A method for learned behavior prediction, comprising:
receiving physiological data from a plurality of different modalities;
grouping the received physiological data by corresponding, similar behaviors;
learning, by a transfer function learner, a shared latent space, in which embeddings from the different modalities are grouped according to the similar behaviors; and
utilizing, by a behavior prediction engine, a trained, transfer function learner to predict an individual's future behavior based on an input of physiological data and a user-specified prediction model.

2. The method of claim 1, in which learning further comprises:
training, for each of the plurality of different modalities, an autoencoder network to convert the received physiological data into a feature embedding in the shared latent space; and
using an association and transfer network to convert the feature embedding from one modality of the plurality of different modalities to another modality for a given behavior outcome.

3. The method of claim 2, further comprising:
computing a transfer loss using explicit cues obtained in a controlled environment to classify whether the received physiological data is obtained from an observed behavior outcome;
computing a second association loss using implicit cues, including whether a time to group the received physiological data from the plurality of different modalities is synchronous or asynchronous; and
optimizing the association and transfer network according to a sum of the association loss and the transfer loss.

4. The method of claim 1, in which utilizing further comprises:
converting, using the transfer function learner, a physiological data signal received from a corresponding modality to another modality of the plurality of different modalities; and
inferring a predictive contribution of a converted physiological data to the behavior prediction engine.

5. The method of claim 4, further comprising leveraging an association and transfer network and the shared latent space to generate an embedding for the physiological signal from one of the plurality of different modalities.

6. The method of claim 1, further comprising:
exploring relationships between physiological data from the plurality of different modalities; and
predicting the individuals future behavior using an available physiological data and an inferred physiological data.

7. The method of claim 1, further comprising:
providing a link to scientific research databases;
exploring, by a user, inferred signals based on an input to the transfer function learner; and
visualizing, by a user interface, the inferred signals.

8. The method of claim 7, further comprising enabling the user to select between the plurality of different modalities to identify spatiotemporal relationships between the inferred signals.

9. A non-transitory computer-readable medium having program code recorded thereon for learned behavior prediction, the program code being executed by a processor and comprising:
program code to receive a physiological data from a plurality of different modalities;
program code to group the physiological data by corresponding, similar behaviors;
program code to learn a shared latent space, in which embeddings from the different modalities are grouped according to the similar behaviors using a transfer function learner; and
program code to utilize a trained, transfer function learner to predict an individual's future behavior based on an input of physiological data and a user-specified prediction model using a behavior prediction engine.

10. The non-transitory computer-readable medium of claim 9, in which the program code to learn further comprises:
program code to train, for each of the plurality of different modalities, an autoencoder network to convert the physiological data into a feature embedding in the shared latent space; and
program code to use an association and transfer network to convert the feature embedding from one modality of the plurality of different modalities to another modality for a given behavior outcome.

11. The non-transitory computer-readable medium of claim 10, further comprising:
program code to compute a transfer loss using explicit cues obtained in a controlled environment to classify whether the physiological data is obtained from an observed behavior outcome;
program code to compute a second association loss using implicit cues, including whether a time to group the physiological data from the plurality of different modalities is synchronous or asynchronous; and
program code to optimize the association and transfer network according to a sum of the association loss and the transfer loss.

12. The non-transitory computer-readable medium of claim 9, in which the program code to utilize further comprises:
program code to convert a physiological data signal received from a corresponding modality to another modality of the plurality of different modalities using the transfer function learner; and
program code to infer a predictive contribution of a converted physiological data to the behavior prediction engine.

13. The non-transitory computer-readable medium of claim 12, further comprising program code to leverage an association and transfer network and the shared latent space to generate an embedding for the physiological signal from one of the plurality of different modalities.

14. The non-transitory computer-readable medium of claim 9, further comprising:
program code to explore relationships between the physiological data from the plurality of different modalities; and
program code to predict the individual's future behavior using an available physiological data and an inferred physiological data.

15. The non-transitory computer-readable medium of claim 9, further comprising:
program code to provide a link to scientific research databases;
program code to explore, by a user, inferred signals based on an input to the transfer function learner; and
program code to visualize the inferred signals through a user interface.

16. The non-transitory computer-readable medium of claim 15, further comprising program code to enable the user to select between the plurality of different modalities to identify spatiotemporal relationships between the inferred signals.

17. A system for learned behavior prediction, the system comprising:
a physiological data collection module to receive a physiological data from a plurality of different modalities;
a physiological behavior grouping module to group the physiological data by corresponding, similar behaviors;
a transfer function learner to learn a shared latent space, in which embeddings from the different modalities are grouped according to the similar behaviors; and
a behavior prediction engine to utilize a trained, transfer function learner to predict an individual's future behavior based on an input of physiological data and a user-specified prediction model.

18. The system of claim 17, in which the transfer function learner is further to train, for each of the plurality of different modalities, an autoencoder network to convert the physiological data into a feature embedding in the shared latent space, and to use an association and transfer network to convert the feature embedding from one modality of the plurality of different modalities to another modality for a given behavior outcome.

19. The system of claim 17, further comprising a user interface to provide a link to scientific research databases to enable a user to explore inferred signals based on an input to the transfer function learner, and to visualize the inferred signals.

20. The system of claim 19, in which the user interface is further to enable the user to select between the plurality of different modalities to identify spatiotemporal relationships between the inferred signals.
